(19) United States
(12) Patent Application Publication
(10) Pub. No.: US 2011/0195430 A1
(43) Pub. Date: Aug. 11, 2011

(54) HIGH PRESSURE ENZYMATIC DIGESTION SYSTEM FOR PROTEIN CHARACTERIZATION

(75) Inventor: Daniel Lopez-Ferrer, Palo Alto, CA (US)

(73) Assignee: BATTELLE MEMORIAL INSTITUTE, Richland, WA (US)

(21) Appl. No.: 13/086,932

(22) Filed: Apr. 14, 2011

Related U.S. Application Data
(63) Continuation of application No. 12/183,219, filed on Jul. 31, 2008.
(60) Provisional application No. 61/026,845, filed on Feb. 7, 2008.
(60) Provisional application No. 61/346,780, filed on May 20, 2010.
(60) Provisional application No. 61/324,218, filed on Apr. 14, 2010.

(51) Int. Cl. G01N 33/53; C12N 9/00; C12M 1/34; C12N 13/00

(52) U.S. Cl. 435/7.1; 435/183; 435/287.1; 435/173.2

(57) ABSTRACT

An on-line method and system for obtaining samples for proteomic analysis that utilizes pressure and a preselected agent to obtain a processing sample in a significantly shorter period of time than prior art methods and which maintains the integrity of the processing sample through the preparatory process, and provides enhanced protein capture. In one embodiment of the invention, a sample and an enzyme are combined and subjected to a pressure, preferably a pressure cycle range that varies between 0 to 35 kpsi, for a period of time of preferably less than 60 seconds. This process results in producing a sample suitable for analysis, which is preferably introduced to another analytical instrument such as a mass spectrometry instrument, or other device.

### PRIORITY

This application claims priority from and is a continuation in part of application Ser. No. 12/183,219 entitled High Pressure Enzymatic Digestion System for Protein Characterization filed Jul. 31, 2008 which in turn claims priority from a provisional patent application entitled High Pressure Enzymatic Digestion System for Protein Characterization, application No. 61/026,845 filed Feb. 7, 2008. This application also claims priority from provisional patent applications 61/324,218 filed Apr. 14, 2010 and 61/346,780 filed May 20, 2010.

### STATEMENT AS TO RIGHTS TO INVENTIONS MADE UNDER FEDERALLY-SPONSORED RESEARCH AND DEVELOPMENT

This invention was made with Government support under Contract DE-AC0576RLO1830 awarded by the U.S. Department of Energy. The Government has certain rights in the invention.

### BACKGROUND OF THE INVENTION

1. Field of the Invention

The present invention generally refers to analytical methods and systems and, more particularly, to the large scale analysis of proteins or proteomics.

2. Background Information

Modern scientific methods in biology have led to a variety of opening technologies, such as genomics, proteomics, metabolomics, which have been utilized to understand relationships and interactions in biological systems. These methods and sciences have also contributed greatly to the advancement of clinical and biotechnological analyses. One of the problems that exists in these disciplines is the time required to prepare and process samples. While various advancements have been made in the reduction of analysis time, one of the key bottlenecks in this process occurs during the sample processing and preparation period. This is particularly true when large scale studies need to be done and consequently a large amount of samples need to be processed. While various schemes have been utilized to attempt to increase the throughput of samples by speeding up the sample preparation process, none of these have been adopted with universal appeal.

In proteomics, the typical sample preparation step, includes the digestion of a complex protein sample, by being incubated with an enzyme, in a buffered medium for a defined period of time, typically overnight or around 12 hours. This extended time requirement slows down the through processing of protein samples and makes protein digestion one of the most time-consuming steps in proteomic analysis workflow. In addition, because such a preparation process is generally carried out manually, associated risk related to operator's error can also negatively impact the analysis. Additionally, manual sample processing can give rise to larger sample/reagent consumption and increased costs due to the labor involved. When working with very small sample sizes which is often the case for clinical applications, automated and quick protein characterization is imperative to limit contamination and other operator-related sources of error, and to bring the use of LC-MS analysis to the next level of efficiency and productivity.

Within the last decade, the development and use of a multidimensional liquid chromatography (LC)-mass spectrometry (MS) based workflow for protein and peptide analysis has benefited biological research as well as, the pharmaceutical, food and biotech industries However even with these advances, sample preparation is typically one of the most time-consuming steps in the analysis workflow as it is typically carried out manually and carries an associated risk of artifacts that include sample contamination (e.g., keratins) and/or irreproducibility. Reducing these sources of error is particularly important for samples that are often only available in limited quantities and sizes, such as from clinical biopsies. Additional drawbacks to manual sample processing are the potential for larger sample/reagent consumption and increased costs due to the labor involved. Enzymatic digestions for example, can take up to several hours to complete, although several methods based on high intensity focused ultrasound have been shown to accelerate the digestion process.

Therefore, what is needed is a method for increasing the rate at which materials such as proteins can be prepared for analysis and analyzed. What is also needed is a method which prepares these materials for analysis which does not negatively impact the analytical workflow utilized therewith. What is also needed is a high throughput system for biotechnological samples that provides efficient, accurate, and precise results in a timely manner. The present invention meets these needs.

Additional advantages and novel features of the present invention will be set forth as follows and will be readily apparent from the descriptions and demonstrations set forth herein. Accordingly, the following descriptions of the present invention should be seen as illustrative of the invention and not as limiting in any way. Various advantages and novel features of the present invention are described herein and will be further be made apparent to those skilled in the art from the following detailed description.

### SUMMARY

The present invention is a method and system for obtaining samples for proteomic analysis that utilizes pressure and a preselected agent to obtain a processing sample in a significantly shorter period of time than prior art methods and which maintains the integrity of the processing sample through the preparatory process. In one application of the present invention, a sample is subjected to a preselected pressure typically somewhere between 0.5 psi and 100 kpsi, for selected periods or intervals of time typically between 5 and 1800 seconds. Through this pressurization process various other agents such as chemicals, enzymes, microwaves, sound, ultrasound, heat, light, and combinations thereof may also be combined with the pressure to affect a desired result and produce a sample having desired characteristics. In other various aspects and embodiments alterations to this basic configuration and protocol can be asserted. For example, in one embodiment of the invention, a sample and an enzyme are combined and subjected to a pressure, preferably a pressure cycle range that varies between 0 to 35 kpsi, for a period of time of preferably less than 60 seconds. In other applications, this method can be embodied in a system for proteomic analysis which includes a sample preparation device that treats a protein sample with pressure and a preselected agent. This sample preparation device is then operatively connected to an analytical instrument, which allows for transfer of the treated sample to the analytical instrument for analysis to take place. In one embodiment of the invention the analytical instrument is a high pressure liquid chromatography (LC) system with a pressurized sample loop. This device may then be coupled to another analytical instrument such as a mass spectrometry instrument, or other device. Various modifications and alterations may be made to the system to perform other tasks such as tagging a process sample with a material such as a radioisotope or other tasks.

In one embodiment the system consists of a flow injection LC system fitted with a pressurized loop to which sample and protease (e.g., trypsin) are simultaneously introduced. The protease effectively and rapidly digests the sample proteins and produces peptides for subsequent MS-based analysis. The reduced analysis time compared to classical methods makes it attractive for high-throughput proteomics. In other embodiments a reactor is also included in such a fast online digestions system (FOLDS) device. In one embodiment of the invention the system also includes a reactor consisting of a capillary containing a stationary phase capable of trapping proteins. This stationary phase can be reverse phase, strong cation exchange, weak cation exchange material, etc. Cell or protein extracts are introduced into the system using a sample loop and then pressurized. Pressure will produce cell lysis or protein denaturation and subsequently proteins will be attached to reactor. To maximize the protein analysis, cysteine residues can be derivatized by, for example, first reducing the protein to free thiols and then alkylating the materials to prevent the thiols from reacting in the future. After this step, proteins would be ready to be digested by adding the appropriate enzyme and adjusting the pH. Once the digestion has finished, peptides can be eluted and analyzed by, for example, a liquid chromatography instrument coupled to mass spectrometer.

While these examples have been provided, it is to be distinctly understood that the invention is not limited thereto but may be variously alternatively configured according to the needs and necessities of a user. The purpose of the foregoing abstract is to enable the United States Patent and Trademark Office and the public generally, especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

Various advantages and novel features of the present invention are described herein and will become further readily apparent to those skilled in this art from the following detailed description. In the preceding and following descriptions the preferred embodiment of the invention, by way of illustration of a best mode contemplated for carrying out the invention have been provided. As will be realized, the invention is capable of modification in various respects without departing from the invention. Accordingly, the drawings and description of the preferred embodiment set forth hereafter are to be regarded as illustrative in nature, and not as restrictive.

### BRIEF DESCRIPTION OF THE DRAWINGS

FIGS. 1a and 1b show the results of testing of one embodiment of the present invention.

FIGS. 2a and 2B show additional results of testing of one embodiment of the present invention.

FIG. 3 shows comparative results of one embodiment of the present invention

FIG. 4 shows additional comparative results of an embodiment of the present invention

FIG. 5 shows various embodiments of the present invention

FIG. 6a-6f show one embodiment of the present invention along with various results and modifications thereof.

FIG. 7 shows various results of one embodiment of the present invention

FIG. 8 shows another embodiment of the present invention

FIG. 9 shows results of one embodiment of the present invention

FIG. 10 shows results of one embodiment of the present invention

FIG. 11a-11d shows results of one embodiment of the present invention

FIG. 12 shows another embodiment of the present invention.

FIG. 13 shows the results of testing performed with the embodiment shown in FIG. 12.

FIGS. 14a-14d show configurations of one embodiment of the present invention.

FIGS. 15a-15c shows another configuration of another embodiment of the present invention.

FIGS. 16a-16b shows the results of the present invention compared to other methods in one embodiment of the invention.

FIGS. 17 a-17d show the results of the present invention compared to other methods in another embodiment of the invention.

FIGS. 18 a-18d show the results of the present invention compared to other methods in another embodiment of the invention.

FIG. 19 shows another configuration of the present invention.

### DETAILED DESCRIPTION OF THE INVENTION

In one embodiment, the present invention is a new method for rapid proteolytic digestion of proteins under high pressure that uses pressure cycling technology in the range of 5 to 35 kpsi w to prepare samples for proteomic analysis, and a system that implements such a method. While these specific examples are shown it is to be distinctly understood that the invention is not limited thereto but maybe variously alternatively embodied according to the needs and necessities of a particular user. In the method and system of the present invention successful in-solution digestions of single proteins and complex protein mixtures were achieved in 60 s utilizing this method and then analyzed by reversed phase liquid chromatography-electrospray ionization ion trap-mass spectrometry. The results of the samples prepared by this method coordinated with the results of samples prepared by the traditional prior art method. However, the method described in the present invention provides greatly simplified sample processing, easy implementation, no cross contamination among samples, and cost effectiveness.

In one set of experiments described hereafter one embodiment of the present invention was compared to a common overnight digestion process. In this particular application, bovine serum albumin (BSA) was used as a standard protein to evaluate the method under different conditions. First, 6 mg of BSA was denatured in 8 M urea and reduced with 10 mM DTT in 25 mM ammonium bicarbonate (pH 8.25) at 37° C. for 1 h. Iodoacetamide was added to a final concentration of 50 mM, and the resulting mixture was incubated at room temperature in the dark for 45 min. Twelve 50-@g aliquots were diluted 4 fold to reduce the urea concentration, using either 25 mM ammonium bicarbonate, 20% MeOH, or 80% MeOH. Trypsin was added (1:50 protease-to-protein ratio), to a final volume of 1.4 mL and the solutions were placed in pulse tubes. The Barocycler™ NEP-3229 instrument and disposable polypropylene PULSE tubes FT-500 were obtained from Pressure BioSciences (West Bridgewater, Mass., USA) and were used for all experiments. The pulse tubes were subjected to the Barocycler™ program, using 4 or 8 pressure pulses for a total of 1 minute per run. Finally, the enzymatic digests were transferred to new centrifuge tubes, acidified, and frozen with liquid N₂ to stop the reaction. The samples were then dried down by centrifugal evaporation and stored at −20° C.

The Shewanella oneidensis, strain MR-1, whole cell protein tryptic digest was prepared by lysing by bead beating, using 0.1 mm zirconia/silica beads in a mini-bead beater for 180 s at 4500 rpm. The lysate was collected and placed immediately on ice to inhibit proteolysis, then denatured with 8 M urea, 25 mM ammonium bicarbonate, 10 mM DTT, (pH 8), and incubated for 1 h at 37° C. Iodoacetamide was added to a final concentration of 50 mM, and the resultant mixture was incubated for 45 min at room temperature in the dark. The mixture was diluted 4 fold, and following the addition of trypsin (1:50 protease-to-protein ratio), was incubated either overnight at 37° C. overnight or for 1 min using PCT at 35 kpsi.

A solution with a final concentration of 1 μM protein in 12.5 mM ammonium bicarbonate was prepared for the myoglobin experiments. Trypsin was added and the samples were digested (1:50 protease-to-protein ratio) during the pressure cycles in the Barocycler™. After treatment, 500 fmol of the protein digest was analyzed by LC-MS/MS. Separations were performed using a 40-nL enrichment column and 43 mm×75 μm analytical column packed with 5 μm ZORBAX 300SB C18 particles. A flow rate of 1 μL/min was employed for enrichment and 600 nL/min afterwards. Peptides were eluted using a 5 min gradient from 5% to 90% Solvent B (0.5% formic acid, 90% acetonitrile; Solvent A: 0.5% formic acid in water:acetonitrile 97:3), with a separation window of ˜2 min. The total analysis time was 12 min. Each sample was analyzed in triplicate. To prevent cross contamination among different samples, a blank was run between each set of replicates.

The data were acquired in survey scans from 500 to 1600 amu (3 microscans) followed by five data dependent MS/MS scans, using an isolation width of 3 amu, a normalized collision energy of 35%, and a dynamic exclusion period of 2 min. MS/MS data were analyzed using Spectrum Mill software against an in-house FASTA database that contained S. oneidensis MR-1 and BSA proteins. Spectra that matched to BSA were manually verified.

For the complex protein mixture analysis, 2 μg of the S. oneidensis digest were analyzed using a custom-built capillary LC system coupled online to a linear ion trap mass spectrometer with an in-house developed ESI source. The LTQ mass spectrometer was operated in a data-dependent MS/MS mode (m/z 400-2,000), in which a full MS scan was followed by ten MS/MS scans, using a normalized collision energy of 35% with a dynamic exclusion of 1 min. Protein identification was carried out using SEQUEST to deduce protein sequences from the S. oneidensis MR-1 genome sequence. Database search parameters included a dynamic modification search (i.e., the presence and absence of the modification was searched) for Met oxidation and a static search (i.e., presence of the modification was searched only) for carbamidomethylation on Cys. Error rates for peptide identifications were calculated as reported previously.

To study myoglobin folding, the protein was directly infused by a syringe pump at 1 @L/min either with or without previous pressure treatment, into an Agilent TOF MS through an ESI interface. MS data were recorded over an m/z range of 500-2500 at a scan rate of 1 scan/sec.

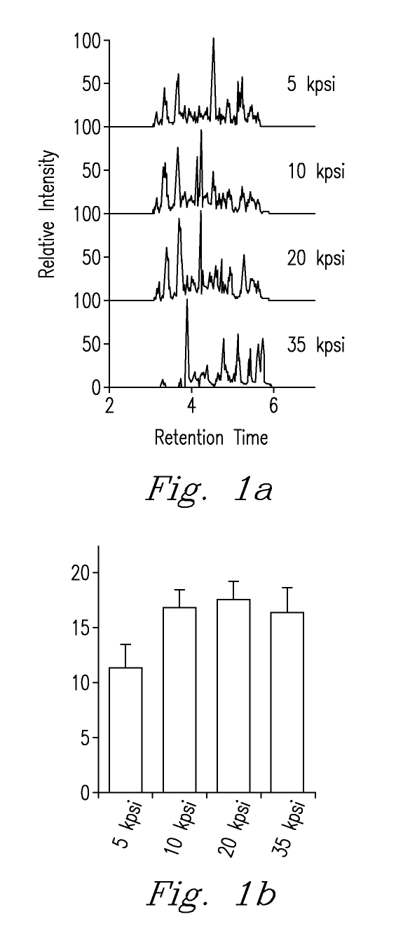

Referring now to FIGS. 1a, and 1b, the effect of enzyme activity in terms of protein proteolytic products at 5, 10, 20, and 35 kpsi for 60 s at each pressure is shown. The chromatograms in FIG. 1a indicate that trypsin activity was not compromised at any of the pressures. However, as is shown in FIG. 1b, digestion of the identified peptides was not as complete at 5 kpsi as those achieved at higher pressures, even though the chromatograms at 5, 10, and 20 kpsi are similar. Although chromatograms belonging to the 35 kpsi samples look significantly different as compare with the others. Manual inspection of the MS spectra showed the same peptides between chromatograms. When pressure was applied to solutions that contained BSA in the absence of trypsin, no protein degradation products were observed, which indicates that the pressure treatment itself did not cause protein fragmentation.

To evaluate the influence of rapid cycling between high and low pressures on trypsin activity, BSA was digested under pressure, using either 4 or 8 differential pressure cycles for a total of 60 s. To further analyze the combined effect of pressure in the presence of an organic solvent for a trypsin digestion, identical BSA protein aliquots were subjected to pressure-digestion at 35 kpsi in the presence of 1) ammonium bicarbonate, 2) an 80:20 (v/v) mixture of ammonium bicarbonate:methanol, and 3) a 20:80 (v/v) mixture of ammonium bicarbonate:methanol. The properties of enzymes in mixed organic-aqueous solvent systems are influenced by factors such as protein structure, presence of phase interfaces, dielectric constants, etc.; all of which contribute to the performance of an enzyme in its biocatalytic system.

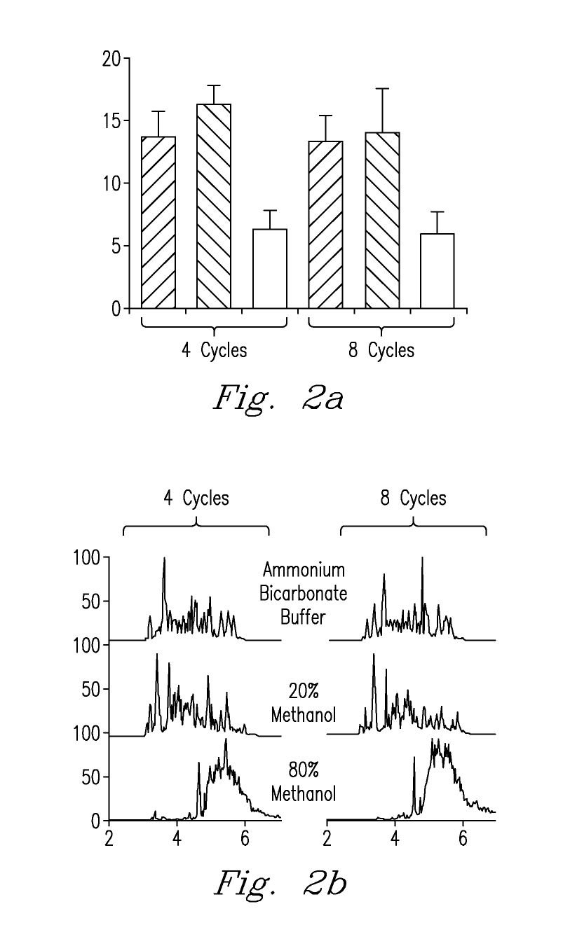

The histogram in FIG. 2a shows that nearly identical results in terms of the number of unique peptide identifications were obtained for samples digested in comparable digestion buffers, regardless of the number of cycles. The chromatographic profiles in FIG. 2b are also very similar for comparable buffers. A comparison of these results obtained at cyclic pressures to those obtained at constant pressure revealed no significant differences, which suggests that at least for trypsin, there is no significant effect in activity due to the number of differential pressure cycles used with PCT during digestion. In addition, the number of identified peptides was not significantly different between aqueous (i.e., ammonium bicarbonate) and 20% methanol digestions for either the 4- and 8-cycle protocols (FIG. 2a).

This embodiment of the present invention was also evaluated by applying PCT to a proteomics sample and then analyzing it using a shotgun proteomics approach. A total proteome extract from a preparation of S. oneidensis cells was separated into two identical aliquots, one of which was subjected to a PCT-assisted digestion at 35 kpsi for 8 cycles during a 60 s time frame and the other, to a trypsin digestion following the conventional overnight approach for comparative purposes. PCT conditions reflected the highest number of cycles and the highest pressure that the Barocycler™ is capable of operating that was shown previously to achieve a good trypsin activity. The digested peptide mixtures were analyzed by reversed phase HPLC using a 100 min gradient.

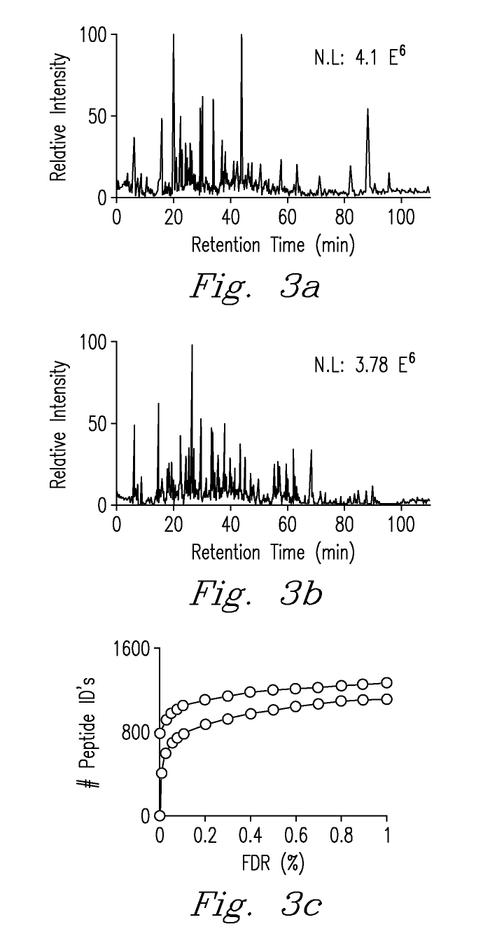

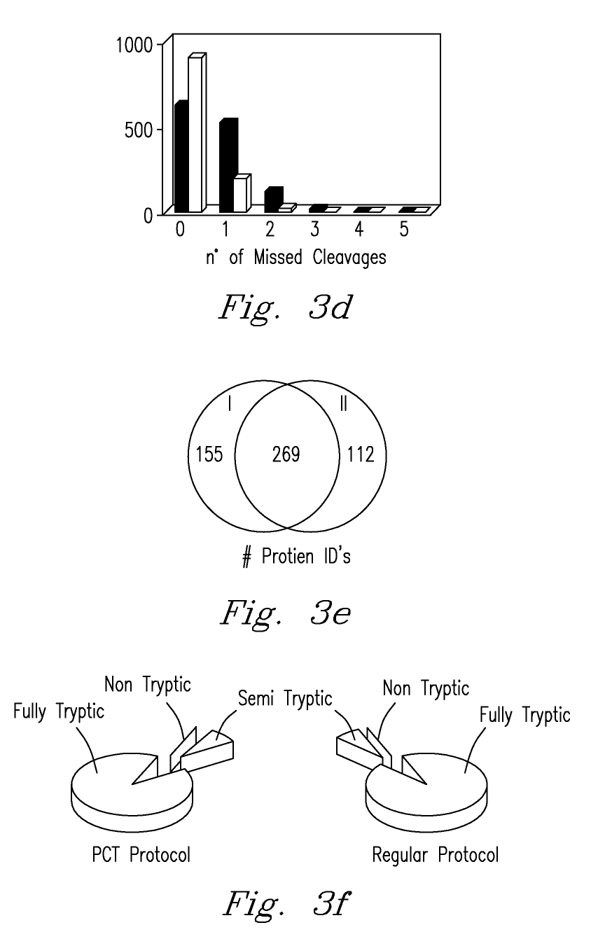

The total ion current chromatograms from the LC-MS/MS analyses of the trypsin digestion using the traditional method at typical ambient pressure and the PCT assisted digestion are provided in FIG. 3 (a and b) for comparison. Note that the chromatograms display very similar intensity profiles in spite of different digestion reactions; however, the total number of identified peptides obtained using the pressure protocol is slightly higher (˜10%) than that obtained by the conventional method (FIG. 3c). The results from a more constrained study of the identified peptides, i.e., false discover rate (FDR)<1% showed that the number of peptides with more than one missed cleavage was much lower for the traditional protocol (FIG. 3d), while approximately more than 95% of the PCT-assisted digested peptides had less than two missed cleavages.

Nevertheless, both experiments had a wide overlap in terms of identified proteins, with the PCT-assisted digestion protocol producing more protein identifications (FIG. 3e). The number of non-tryptic peptides was insignificant in both samples (FIG. 3f) and within the error limits we set for identifying peptides (i.e., <1% FDR). The population of semi-tryptic peptides, defined as a peptide with one end that is not tryptic, is very similar for both digestions.

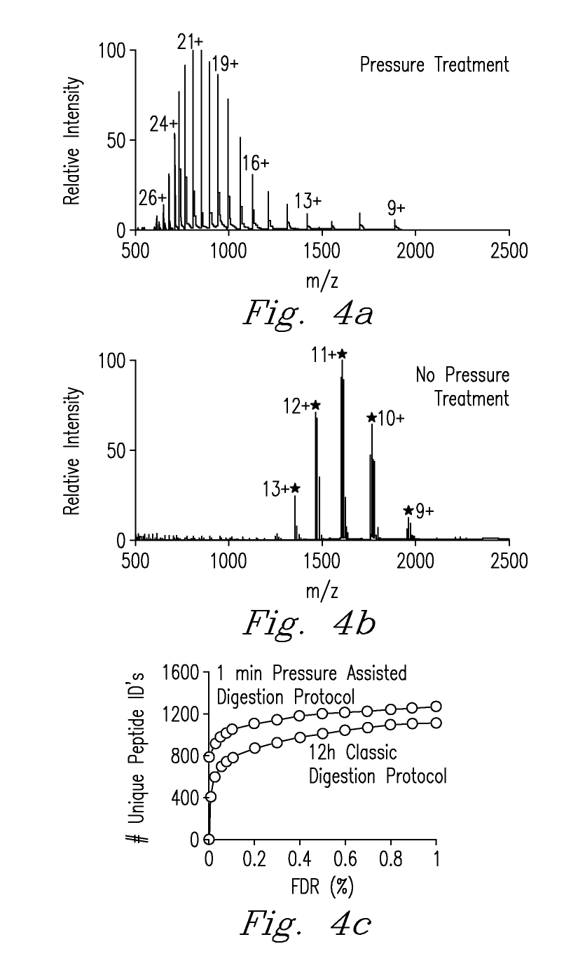

Finally, to test the hypothesis that a better digestion yield is due to the unfolding effect caused by pressure, myoglobin was dissolved in 12 mM ammonium bicarbonate and subjected to 10 kpsi for 60 s prior to direct infusion into the mass spectrometer. For comparative purposes, a native myoglobin protein sample not subjected to pressure treatment was also analyzed by TOF-MS. As shown in FIG. 4, the spectra for pressure-treated and non-pressure-treated proteins taken at a neutral pH are completely different. Remarkably, the charge states corresponding to the native protein are much lower than those corresponding to the pressure treated protein. The native protein yielded the maximum signal at a MW of 17,568 Da, whereas the distribution of the charge states for the pressure-treated protein shifted to higher charge states, and the MW corresponded to 16,952 Da. This finding indicates a loss of the heme group (−616 Da) and complete denaturation of the protein due only to the pressure treatment; unfolding of the native protein permitted a reduction of charge-charge repulsion forces, which in turn allowed a larger degree of protonation.

These results demonstrate how an increase in pressure can dramatically increase the rate of the enzymatic digestion of proteins in proteomic samples. Among the advantages afforded by the present invention include, automated sample preparation, high sample throughput (up to three samples per minute in our setup) without compromising the digestion yields, high reproducibility, no aerosolization (a common effect that occurs when HIFU is applied), and the acquisition of results comparable to those obtained using regular digestion protocols but in a much shorter time-frame (i.e., 1 min). Since digestions can be completed at 20° C., undesired protein modification can be avoided.

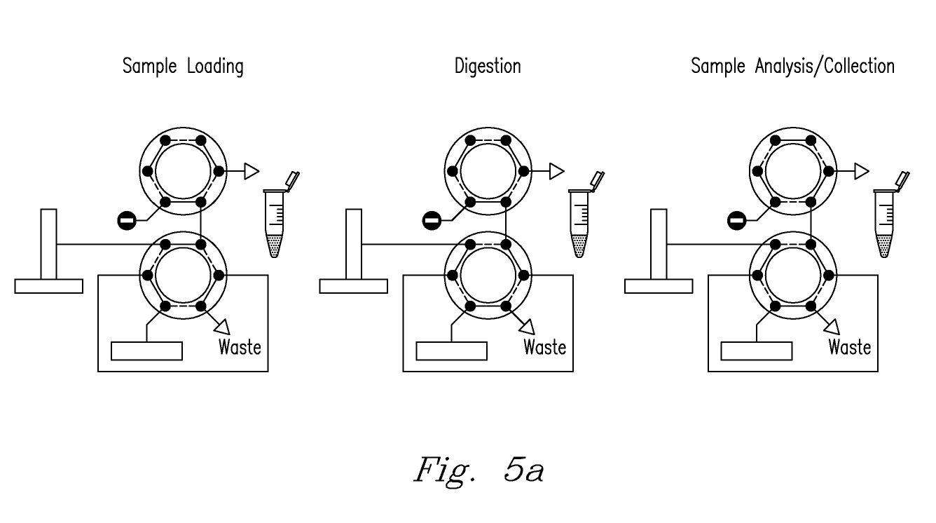

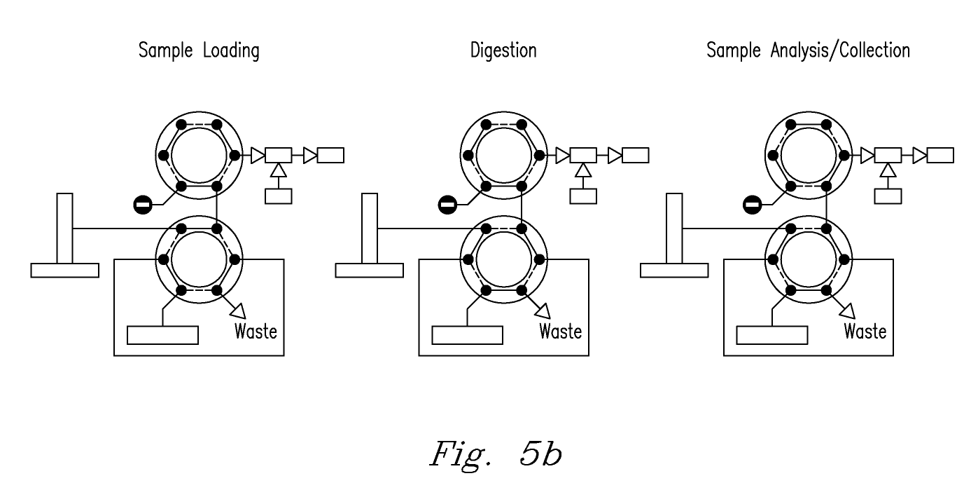

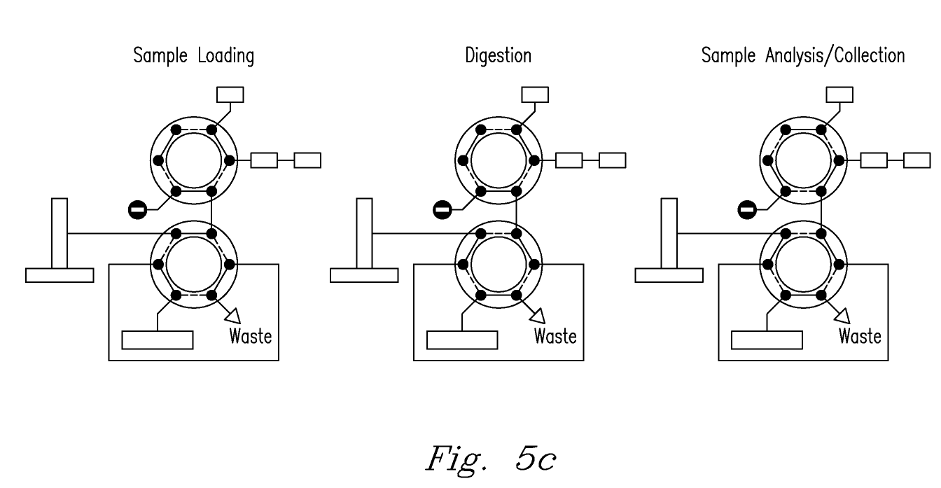

The methods of the present invention can be implemented through systems such as the systems shown in FIG. 5a-5c. FIG. 5a shows an off-line system, while FIGS. 5 b and 5c show on-line digestion systems that reduce the number of sample manipulation steps for high throughput proteomics. In this embodiment, a pressurized sample loop is included in a liquid chromatography-based separation system wherein both sample and enzyme (e.g., trypsin) can be simultaneously introduced to produce a complete, an ultra-fast digestion. In this embodiment the fluidic components of the system consist of a 6-port injection valve with a 5 μL sample loop, and a 4-port valve that are rated to 15,000 psi. A 10,000 psi syringe pump was used to supply mobile phase to the system. The fast on-line digestion system (FOLDS) was operated at a constant pressure of 7,000 psi and used water as a mobile phase. Several modifications where implemented to couple the FOLDS on-line to a mass spectrometer. In the embodiment shown in FIG. 5b, a second syringe pump filled with 90% acetonitrile and 1% formic acid was used to re-acidify the sample just prior to ESI. In the third configuration, the FOLDS was coupled to an Agilent LC 1100 system equipped with a nano-flow pump. Peptides were eluted using a gradient from 10 to 60% solvent B (Solvent A: 0.5% formic acid. Solvent B: 0.5% formic, 80% acetonitrile).

The operation of the FOLDS and details showing valve, port, and sample loop placement are shown in FIG. 5. System functionality is described in three parts, corresponding to stages of sample processing: loading, digestion, and analysis or collection. FIG. 5 illustrates that initially during the sample loading stage the loop is filled with 5 μl of sample and dissolved trypsin. To begin the accelerated protein digestion, the first valve is switched to inject position that enables system pressurization to 7,000 psi, but the liquid flow to the rest of the system is blocked since the second valve is in the load position and the port is closed. In the digestion stage, the sample loop becomes a reaction chamber and digestion is allowed to continue for 1 to 3 minutes. When the pressure-assisted digestion is finished, the second valve is switched to the inject position, initiating the sample analysis stage. The digested sample is either directly infused into a mass spectrometer, collected for off-line analysis, or directed to a reversed phase column for chromatographic separation.

Initially the ODS was employed in an off-line mode (FIG. 5a) to characterize digestion efficiency. Once the samples were collected and dried down, they were resuspended in 10 μL of 40% MeOH:Water, 0.1% formic acid buffer and electrosprayed using a TriVersa Nanomate into a modified Bruker 12 T APEX-Q FTICR mass spectrometer, as previously described¹¹. Each mass spectrum was recorded every 2 s, and an average of three mass spectra was used for data analysis. For on-line applications, two different set ups were used. As shown in FIG. 5b, the FOLDS was coupled to a platform incorporating a home-built IMS apparatus with an Agilent 6210 oTOF MS. In another set of experiments, the FOLDS was coupled to a capillary RPLC separation followed by an ion trap and operated as previously described (FIG. 5c).

Proteins were solubilized in a 12.5 mM ammonium bicarbonate buffer (pH 8.2) and mixed with the sequencing grade modified trypsin, in a 1:50 enzyme-to-substrate ratio. The mixture was then loaded into the pressurized system. Digests were either collected for off-line experiments or analyzed directly using MS. Bovine serum albumin was first reduced with 10 mM DTT at 37° C. during 1 hour and alkylated with 50 mM IAA at room temperature for 45 min. The Shewanella oneidensis was prepared as described elsewhere and used as a control. Otherwise, the soluble proteome was prepared in the same way as described above.

For those analyses where only high resolution MS was employed, protein identifications were carried out using a MASCOT search engine. Due to low complexity of the samples, if the score was outside of the uncertain zone the protein was considered identified. For the MS/MS analysis, a SEQUEST™ database search engine was used. For calculation of the error rates associated with peptide identifications, the same method as published before was used.

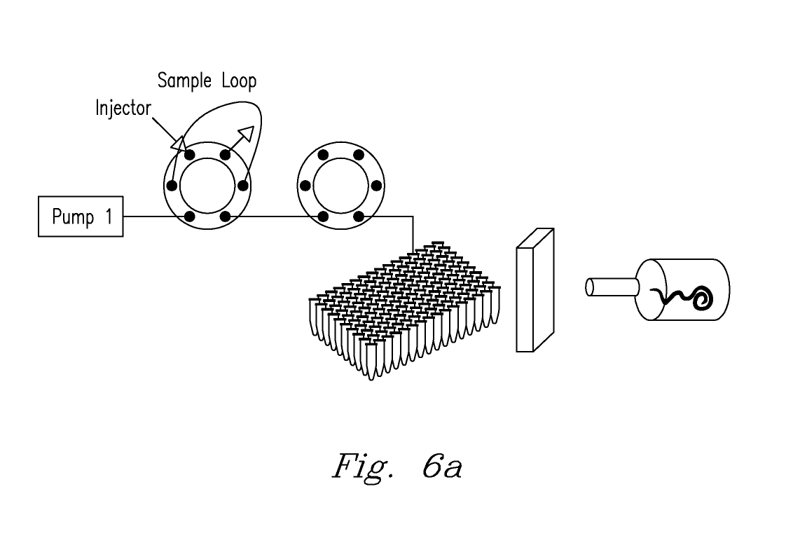

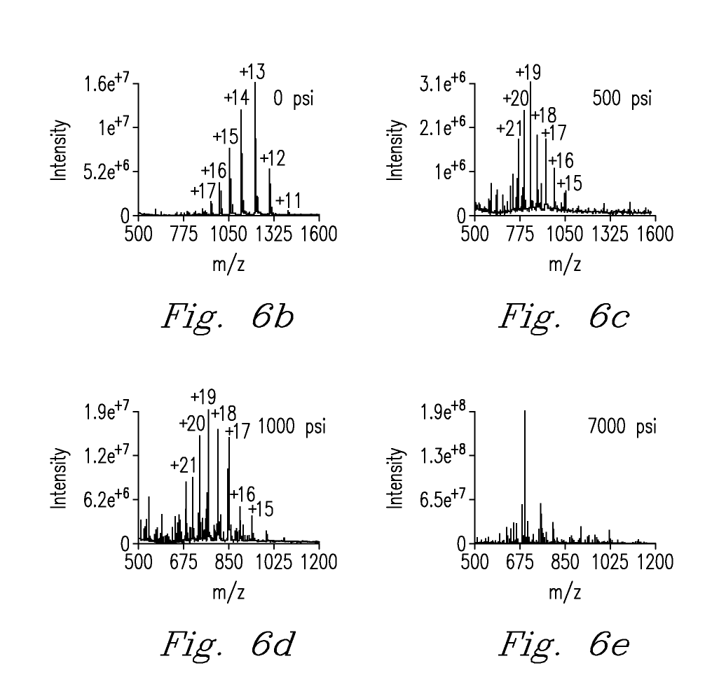

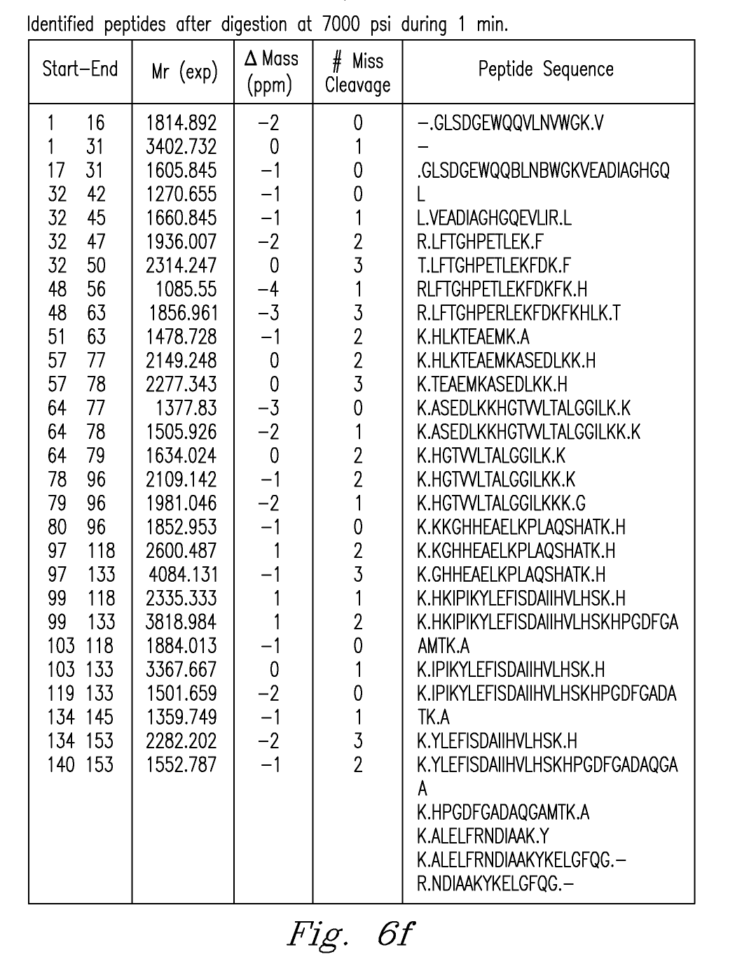

The first experiment used 1 pmol of myoglobin in 12 mM ammonium bicarbonate with trypsin that was injected into the FOLDS (FIG. 5a). The pressures were applied during 1 min and varied from 0 to 7000 psi. After a minute, the sample was collected in a reaction tube and analyzed using ESI-chip-assisted direct infusion into the 12T FTICR MS (FIG. 6). The use of FOLDS allowed us to simultaneously detect any non-digested intact proteins along with proteolytic peptides. When no pressure was applied, the protein eluted from the FOLDS was detected with charge states corresponding to 11 to 15. When pressure in the FOLDS was increased to 500 psi, higher charge states for the protein, but no peptide fragments, were observed. This is most likely due to a dramatic change in the protein tertiary structure resulting in previously unexposed sites being protonated. In the third experiment, pressure was increased to 1000 psi. The MS analysis revealed that digestion and denaturation processes began to occur, showing a mixture of both peptides and the intact protein. The peptides produced and identified at this pressure provide 100% protein coverage with less than three missed cleavages. Finally, in order to confirm a correlation between higher pressures and a digestion rate, we increased the pressure in the FOLDS up to 7000 psi. As a result, complete protein digestion was achieved in one minute, as shown in FIGS. 6b-6f.

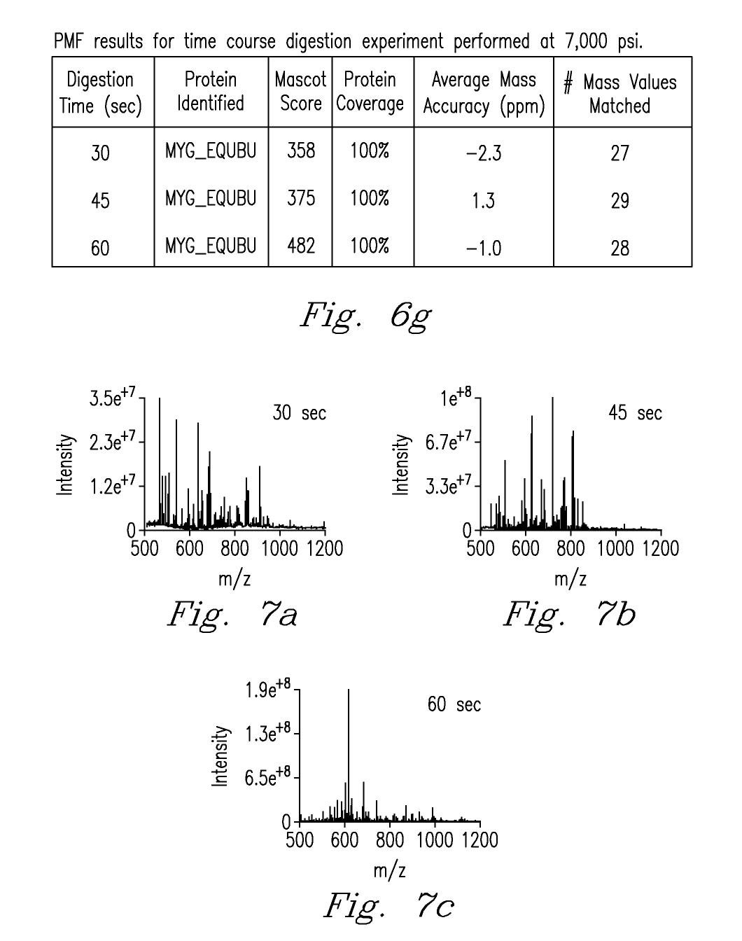

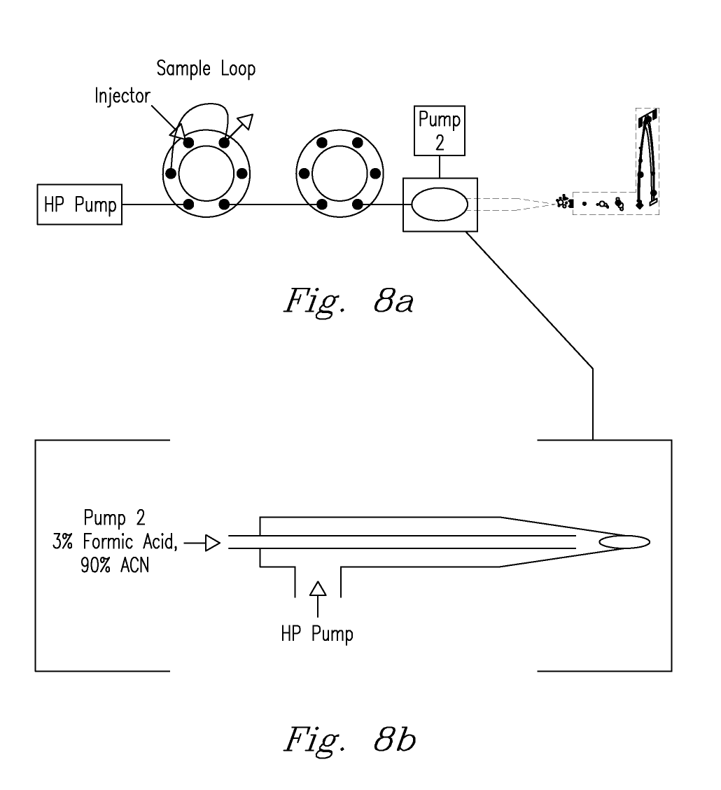

Since 7000 psi pressure facilitated complete and rapid digestion, we further explored digestion kinetics. FIG. 7a-7c shows that even at 30 sec reaction time a satisfactory digestion was achievable. By analyzing peptides generated in 30 sec digestion with the high mass accuracy 12 T FTICR MS, we were able to obtain good protein coverage but with a considerable level of missed cleavages.

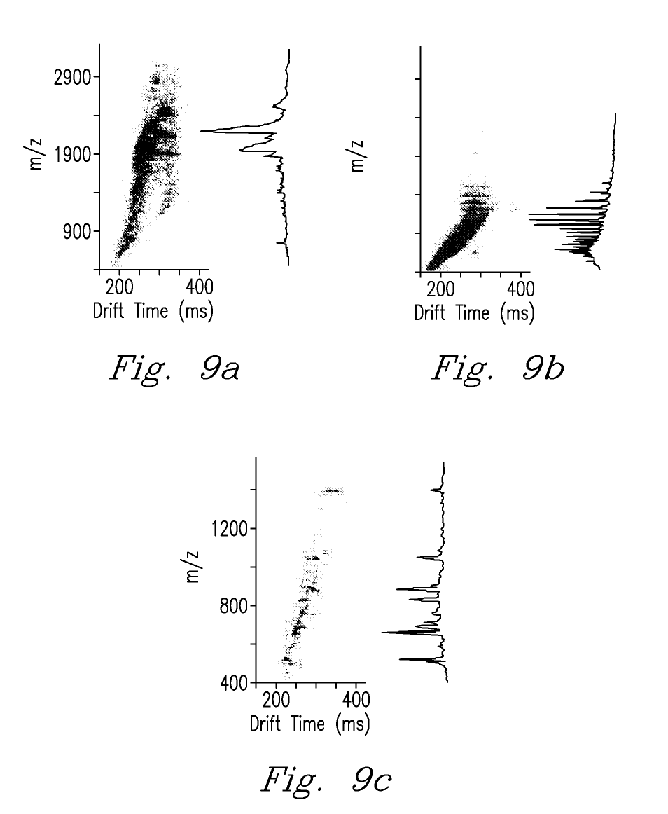

To increase the sensitivity of the device, an organic buffer (90% MeOH, 1% Formic acid) delivered by an independent syringe pump was mixed with the FOLDS eluent at the ESI emitter yielding an improved ionization efficiency. Back flow of the acidified solvent was prevented by the higher pressure of the FOLDS pump. A schematic view of Myoglobin digestion was investigated, using one minute pressure application with and without trypsin. FIG. 9 shows that the myoglobin digestion results obtained with the IMS-TOF-MS were consistent with those reported for FTMS. The total analysis time from the start of injection until spectrum acquisition was less than 90 sec. The key contributor to this speed gain is the ability of the IMS-TOF MS to separate ions quickly in the gas phase based mainly on their charge and gas-kinetic cross section.

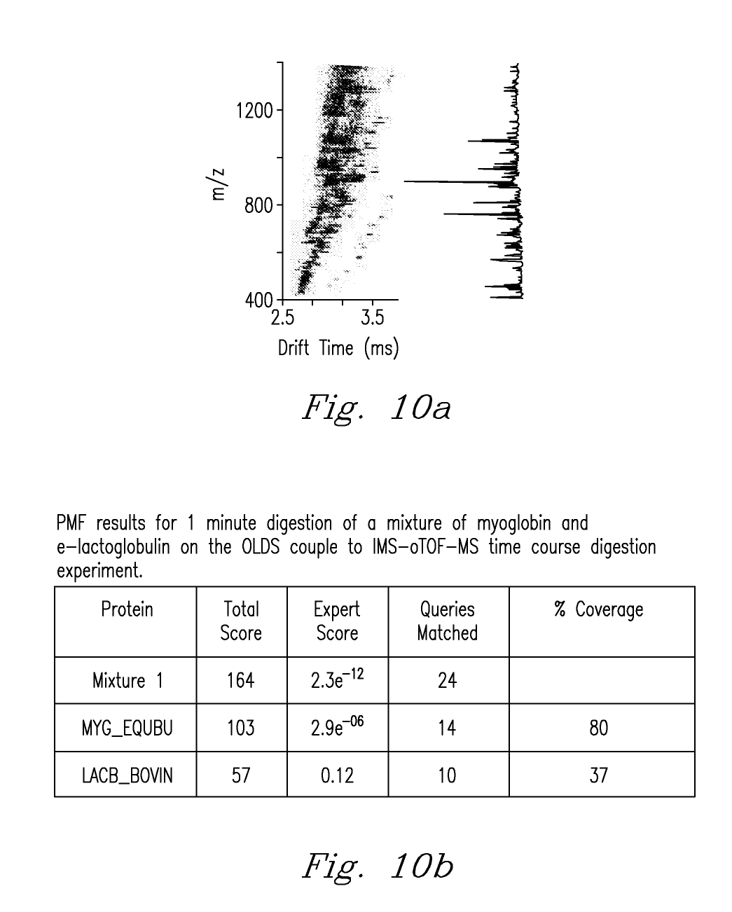

FIG. 10 shows the result of analysis of an equimolar mixture of myoglobin and β-lactoglobulin (1 pmol each) using the FOLDS coupled to the IMS-TOF MS. The mixture was digested under the elevated pressure for one minute, and the products were delivered to the IMS-TOF MS as described above. The peptide mass fingerprinting analysis yielded identifications of both proteins with high MASCOT scores as well as high protein coverage, as summarized in the table found in FIG. 10b. One of the disadvantages of the ODS in the on-line direct infusion configuration is that proteomic sample processing often requires the use of salts and chaotropes, which can generate increased chemical noise and reduce ionization efficiency that impairs accurate protein identifications.

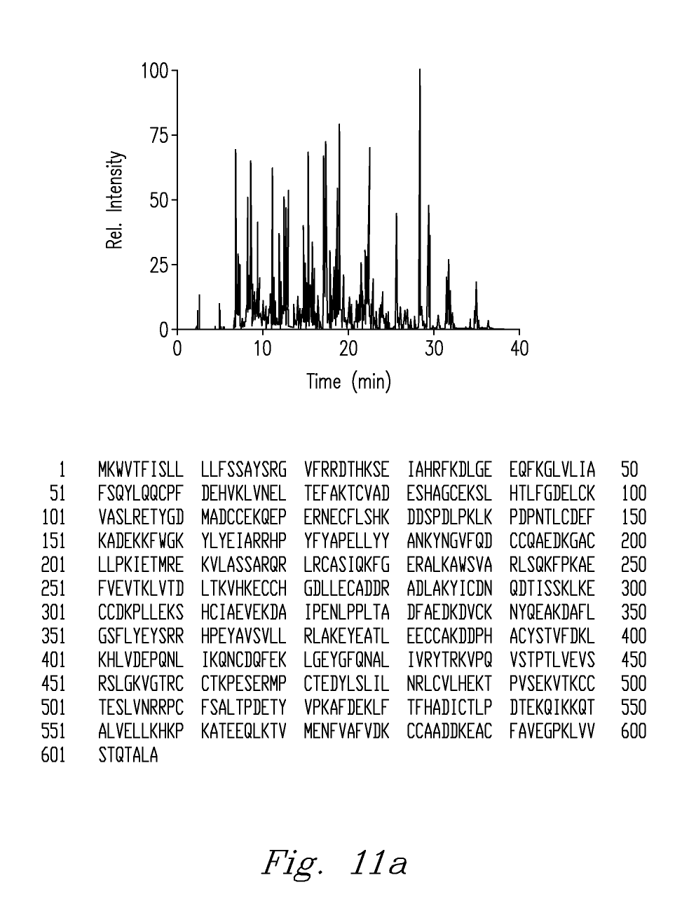

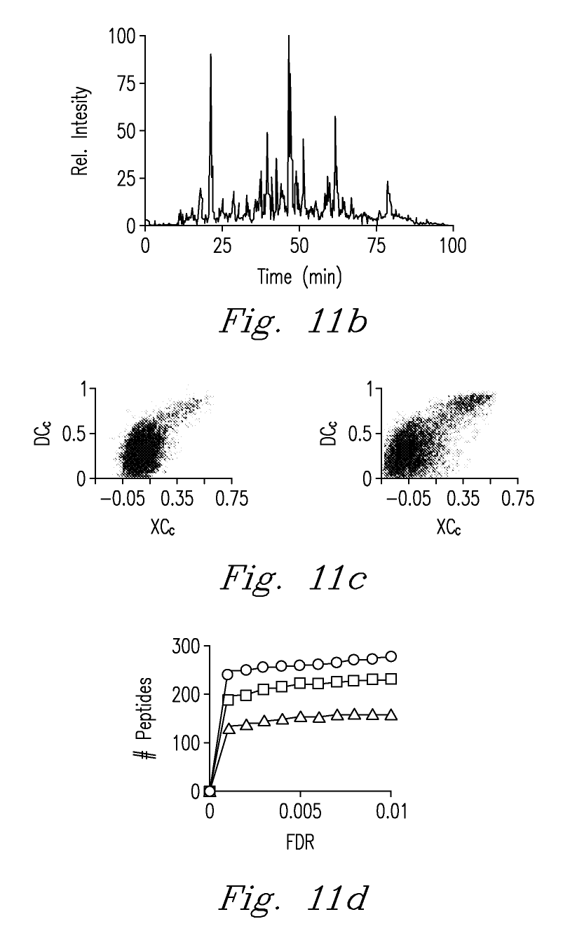

To address this limitation and achieve higher sensitivity, we coupled the FOLDS to RPLC separation to desalt the post-digestion sample. In this study, 10 pmol of BSA was reduced and alkylated in 8M urea, diluted 10-fold with 12.5 mM ammonium bicarbonate, and mixed with trypsin. 1 pmol of the resulting solution was injected into the FOLDS. After a 1 minute digestion at 7,000 psi, the peptide products were loaded onto a reverse-phase column at a flow rate of ca. 10 μL/min and then separated using a 20 min gradient under the conditions described in the Experimental section. The LC effluent was analyzed with an LCQ mass spectrometer in MS/MS mode. FIG. 11a shows that high protein coverage, ca. 70% of the amino acid sequence, was achieved and the 48 identified peptides were all homogenously distributed across the protein sequence.

In one experiment, a bacterial proteome of Shewanella oneidensis was studied. The soluble proteome was resuspended in 25 mM ammonium bicarbonate, trypsin was added in a 1:50 enzyme:protein ratio and 5 μg of total protein amount was injected into the system and digested at 7,000 psi for 1 minute. Proteolytic digests obtained using the conventional protocol and the FOLDS were analyzed for comparison with and without reduction/alkylation procedures. In the conventional proteome digestion with trypsin, a 5 μg aliquot of Shewanella oneidensis was subjected to regular digestion for 5 hours at ambient pressure with no chaotropes added and without reduction or alkylation of the proteins. The same sample was then digested with the FOLDS without chaotropes and reduction/alkylation. As a result, the number of identified peptides with the conventional procedure was found to be lower than that identified from the FOLDS-processed sample.

Since the previous myoglobin studies had shown that pressure denatures proteins, concurrently accelerating reaction kinetics, a second set of experiments was aimed at evaluating the effect of denaturation. A 5 μg of the Shewanella oneidensis proteome was reduced and alkylated in the presence of 8 M urea and then subjected to trypsin digestion using both the conventional and FOLDS protocols. FIG. 11b shows that the number of identified peptides with the conventional procedure was close to those obtained with the FOLDS. This study indicates that not only the increased pressure accelerates digestion rates also because acts as a denaturing agent without the need of adding chaotropes, facilitating the formation of the complex between the enzyme molecules and the substrate. Furthermore, these data and the structural changes observed for myoglobin treated at high pressure suggest that not only does increased pressure accelerate proteolytic digestion, but it also denatures the protein like a chaotrope. Further research is necessary to confirm this hypothesis. Nevertheless since pressure denatures proteins, but trypsin is still working, we believe that sequencing trypsin is engineered on the way that its catalytic triad is tremendously stable to high temperature and in the same way to pressure.

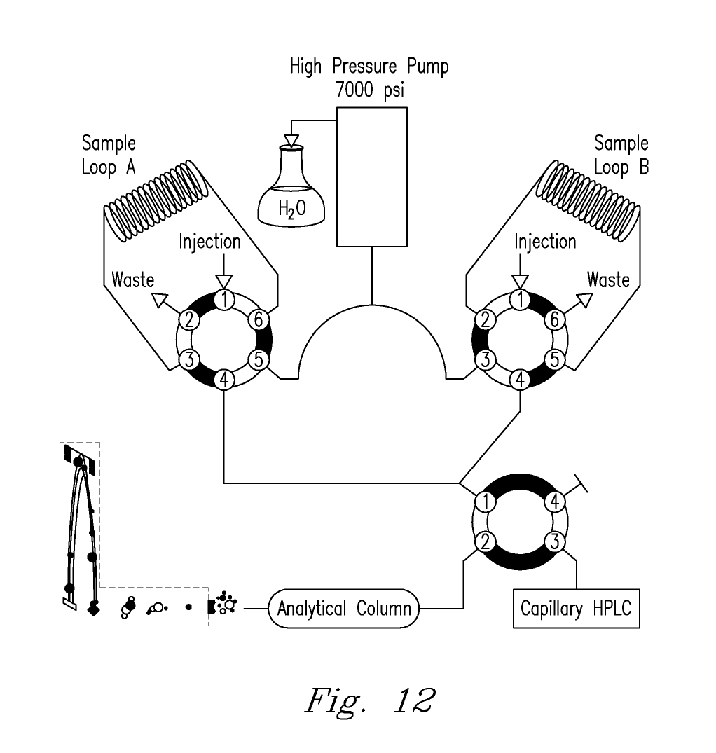

In another set of experiments, we demonstrate the possibility of isotopically labeled peptides using a dual on-line digestion system by changing regular water for ¹⁸O enriched water in the digestion buffer FIG. 12. This figure shows an enzymatically catalyzed reaction where all generated peptides can be labeled with at least one ¹⁸O atom to a maximum of two in the carboxy terminus. The reaction is described in the scheme above where a protease incorporates two O-18 atoms by reversible binding of peptides by enzyme molecules of the serine protease family.

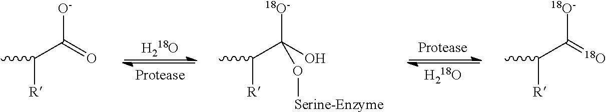

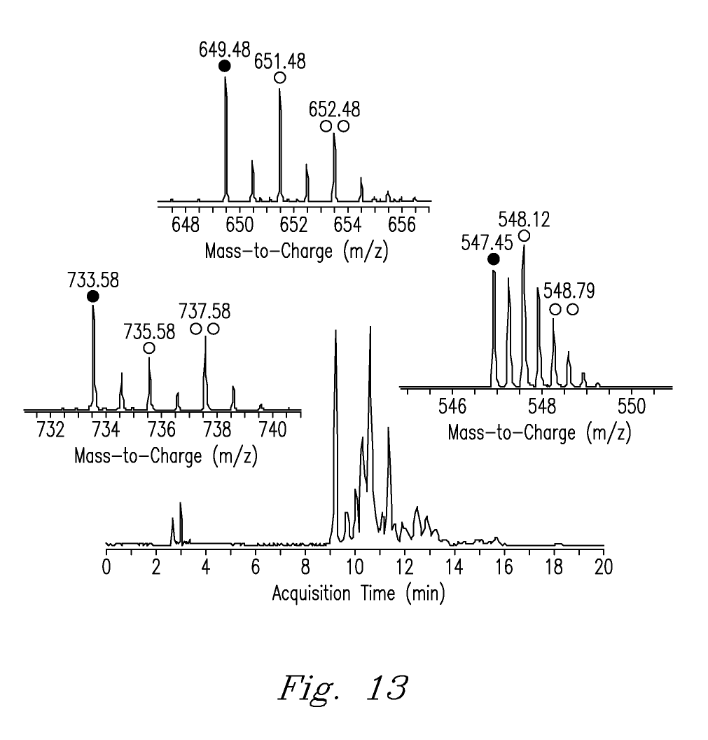

Introducing stable isotopes allows global quantitative comparisons in between different samples due to the mass differences that isotopes introduce in each sample. As proof of concept, FIG. 12 shows an easy set up were to on-line protein digestions can be performed simultaneously. Two equal aliquots of BSA protein have been on-line digested, isotopically labeled and analyzed using LC-MS. The results obtained with the optimized workflow incorporating FOLDS-enhanced digestion and labeling are comparable to those obtained using traditional, more time consuming proteomics workflows, indicating that data quality is not compromised by going faster. This dual FOLDS system may provide significant improvement in the overall protein analysis throughput for biological applications involving large numbers of samples, such as for clinical studies of biomarker discovery. FIG. 13 shows some of the peptides generated in the presence or absence of 18O water. The ratio between light and heavy isotopes can be translated to the ratio of a given protein in two different samples. This experiment can also be simplified by doing sequential digestions, where the products of the first digestion are trap in the analytical column and the second digestion takes place. When this last one finishes, the peptides are flush to the analytical column, where they will be storage together with the peptides of the prior digestion.

The use of FOLDS in conjunction with MS for the identification of digestion products has accomplished several objectives. First, extended incubation times are no longer needed for effective protein digestion since the application of high pressure accelerates the proteolysis kinetics. Second, the use of trypsin in solution eliminates non-specific binding observed with immobilized enzymes. Third, the coupling of the FOLDS to IMS-TOF MS yields an analysis platform with the capacity to rapidly detect large numbers of peptide ions in an extremely fashion way very useful in single protein characterization or monitoring. This peak capacity can be further increased by coupling capillary RPLC separation to the IMS-TOF MS instrument. In all of the configurations reported, many sample handling steps are eliminated, making the automation of these methods feasible.

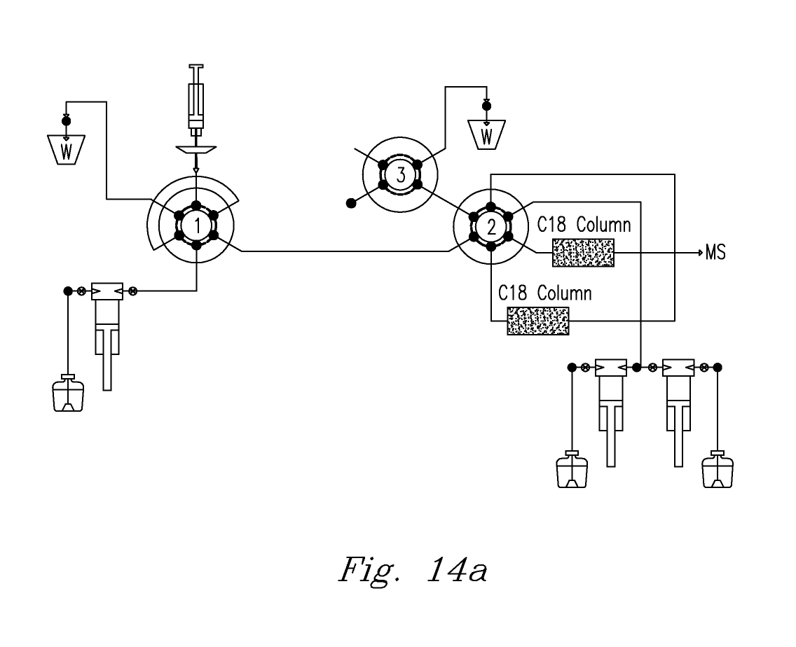

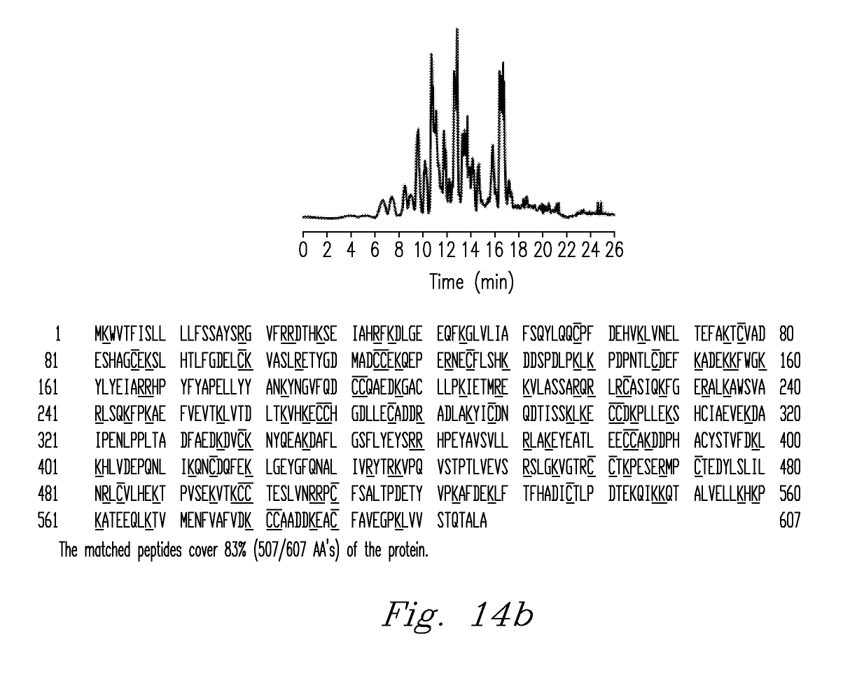

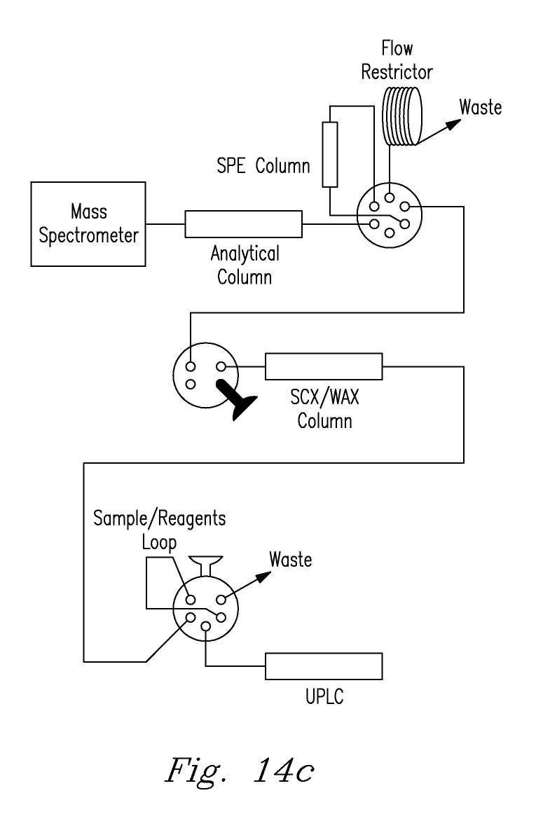

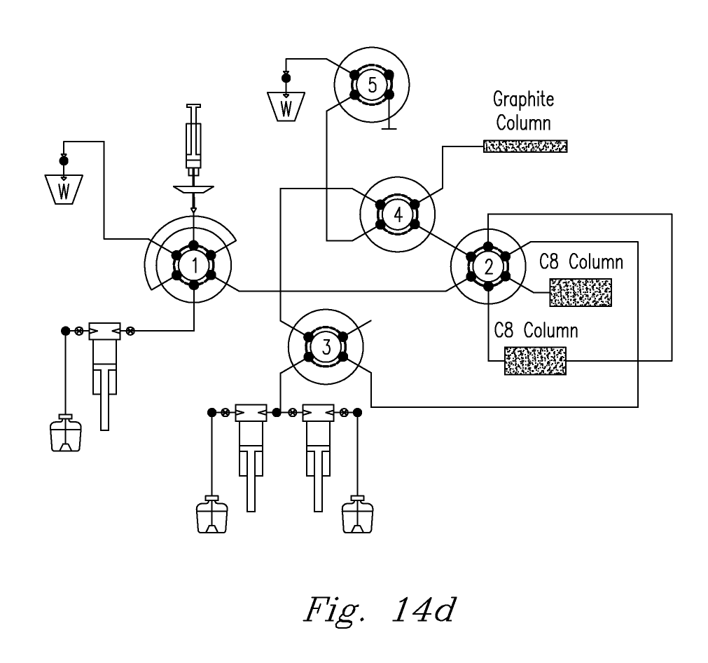

In one embodiment of the present invention the four stages of sample processing: Loading, derivatization, digestion and analysis can be configured in a single application. An example of such a configuration is shown in FIG. 14A. FIG. 14 A shows schematic of a simple setup for a Fast On-Column Digestion System. The components include a syringe pump, two six port valves and a four port valve and an LC-MS system. A RPLC separation column was coupled to the second six port valve and an ion trap was used for signal detection.

During the sample loading, the loop is initially filled with 5 μL of sample. This volume was kept fixed for the further additions of the different reagents. To initiate cell lysis, the first valve is switched to the inject position to pressurize the system to 10,000 psi. The liquid flow to the rest of the system is blocked since the third valve is in the load position and the port is closed. The released proteins are captured in the column and then TCEP and IAA are added. In this case, valve 3 is open allowing to trap the reagents on the head of the column with the rest of the proteins. Valve 3 is against closed allowing to pressurize the whole system at 10,000 psi and avoiding the reagents to leave the system since they will not be retained by the reactor. Valve 3 is open again to wash the proteins from the excess of reagents. In the digestion stage, the enzyme is introduced into the system through the sample loop. Valve 3 will be open, in this case, to trap the enzyme molecules on the head of the column with the rest of the proteins. Digestion will take place once the system is pressurized again and digestion is allowed to continue for 1 to 3 min. When the pressure-assisted digestion is finished, the second valve is switched to coupled the reactor column to the analytical column to initiate the sample analysis stage.

In order to prove the feasibility of the system bovine serum albumin was chosen as standard protein. The high amount of disulfide bridges typically makes digestion of this protein particularly challenging. However, after reduction and alkylation with tris(2-carboxyethyl)phosphine (TCEP) and iodoacetamide (IAA), the sample was washed and pepsin was added in a ratio 1:25 enzyme substrate. The system was pressurized for 5 min at 10000 psi to allow the digestion to occur. Peptides were then eluted and separated using a linear gradient and analyzed by coupling the system to a mass spectrometer. FIG. 14B shows the base peak chromatogram of the BSA digest and the protein sequence coverage under the following conditions: 1 μg of BSA was loaded onto the column through the sample loop. Then 10 mM TCEP and 100 mM IAA was introduced in the reactor, the flow at Valve 3 was monitored to ensure that the reactor is full of the denaturating solution, the reaction took place over 10 min. Proteins were washed and pepsin was added (1:25 Enzyme:substrate ratio) and trapped in the C8-SPE column together with the BSA. Digestion was performed at 10000 psi for 5 min. Peptides were analyzed using a 1200 Agilent HPLC at 1.2 mL/min connected on-line to an LTQ-FTICR mass spectrometer. No remaining protein was observed during the run

In addition to this embodiment other alterations and modifications may also be made to the system. For example in FIG. 14C a configuration is shown which allows for cell lysis reduction and alkylation, protein digestion and 2D peptide chromatography for bottom-up analysis. In this configuration an SCX column is included which will allow a further fractionation of the digested peptides and allow for two dimensional peptide chromatography. In operation cells are loaded into the system, and the system is pressurized. The proteins are then trapped on the capillary and washed. TCEP (tris(2-carboxyethyl)phosphine) and IAA (iodoacetamide) are added to reduce and alkylate the proteins. Trypsin or Pepsin are then added, trapped onto the reactor and pressurized. The peptides are released from the reactor by increasing the concentration of salt or can be fractionated by doing salt steps. The resulting peptides are then analyzed by LC-MS/MS. In an alternative configuration such as the one shown in FIG. 14 C an RP-SPE column is substituted for the SCX/WAX column and a similar process is utilized. However in this subsequent configuration the release from the reactor is not performed by increasing the concentration of sale or fractionated by doing salt steps. This results in a simple and straightforward means for performing 1D peptide chromatography.

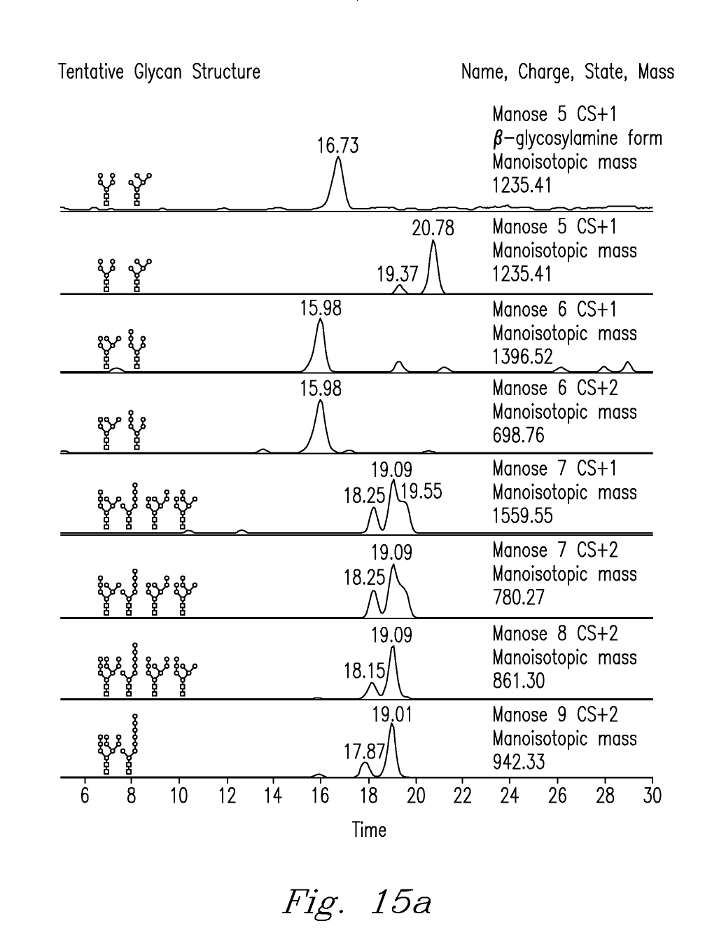

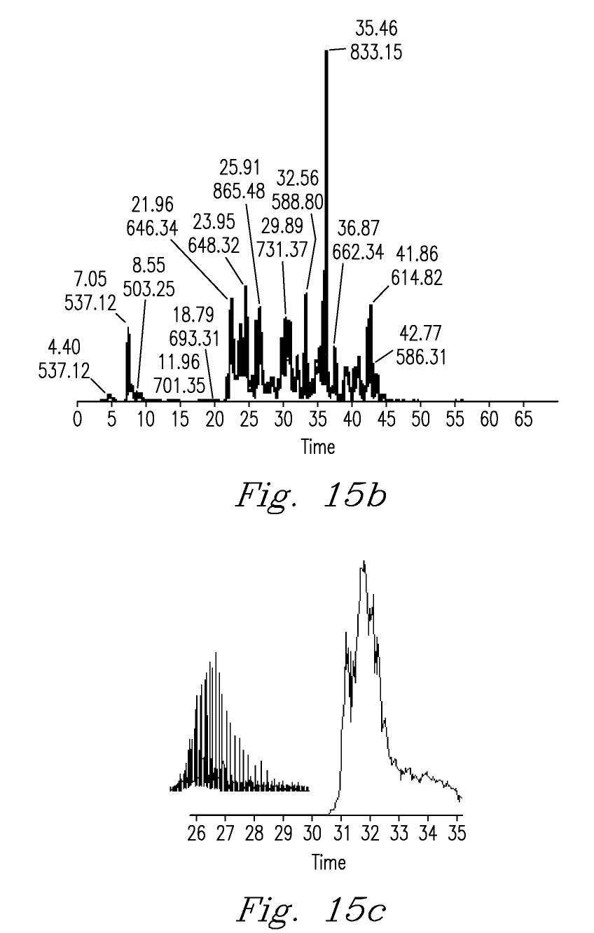

FIG. 14C show a configuration for an embodiment that allows for sequential enzymatic reactions, for example, a first digestion can take place in the loop and then a second digestion can be done in the column. Multiplexing is also possible since while some of the target molecules are being analyzed other reactions can take place. As a example of this feasibilities, a system is described that allows protein deglycosylation, glycan analysis (FIG. 15a), and further analysis either of the peptides originated from the protein digestion (FIG. 15b) or the intact deglycosylated protein FIG. 15c. Glycosylation is of great importance for many recombinant protein drugs, with over one-third of recombinant protein drugs being glycoproteins. There are typically three steps in glycan profiling analysis: (1) Release of glycans from glycoproteins (2) separation of glycans, (3) detection, identification, and quantitation of released glycans.

One of the most important goals nowadays in the field of proteomics/glycomics is to be able not only to perform glycan profiling, but also indentify the glycosylated protein and identify the glycosylation site. And the only way of being capable to achieve this goals in to integrate both types of analyses. By using the fast on-column digestion system this is possible. The system integrates the following functionalities: (1) glycan release with solution PNGase F, (2) capture of the released glycan, (3) Glycan separation based on graphitized carbon chromatography, (4) nanoelectrospray into a mass spectrometer for glycan detection and quantitation, and at the same time (5) protein digestion and (6) peptide separation on a reverse phase column.

FIG. 14c shows an alternative schematic diagram of an embodiment of the application wherein Fast On-Column Digestion System for integrating glycomic and proteomic analyses can be achieved. The principal components include a syringe pump, two six port valves and three four port valves and an LC-MS system. In one embodiment a RPLC separation column was coupled to the second six port valve and graphite column to the third valve. A LTQ-FTICR mass spectrometer was used for signal detection.

To characterize the utility of the system, bovine pancreatic ribonuclease B was processed through the system. This protein has a single N-glycosylation site at Asn34, where five to nine mannose residues can be attached to the chitobiose core. In this system on-line deglycosylation and protein removal, glycan capture and glycan separation took place. The deglycosylation was carried out using PGNase F at 10000 psi on the injection loop. Deglycosylated protein was then capture in a C8-SPE column (200 mm ID×50 mm OD). Glycans were capture in a graphite column (100 mm ID×150 mm OD). Glycan analysis was performed using a 1200 Agilent HPLC at 1.2 mL/min connected on-line to an LTQ-FTICR mass spectrometer. From this testing it was determined that pressure was able to cleave the sites in an accelerated fashion and could be coupled to an analytical platform. The major advantage of this system is that by using C8 as stationary phase for the reactor, proteins are trapped while glycans are easily released and trapped on the graphite column. After the glycan analysis, the entrapped protein can be digested or directly eluted to measure its MW.

FIGS. 15A, 15B and 15C show the results after glycan analysis comprising either a further protein digestion or analyzing the intact protein. FIG. 15 A shows the profiling of the high mannose glycans release after PGNase F digestion. FIG. 15B shows the base peak chromatogram from bovine Ribonuclease B digest obtained after deglycosylation. Pepsin was added (1:25 Enzyme:substrate ratio) and trapped in the C8-SPE column together to the deglycosylated protein. Digestion was performed at 10000 psi for 30 min (note that while the glycans are being analyzed protein digestion can take place). Peptides were analyzed using a 1200 Agilent HPLC at 1.2 mL/min connected on-line to an LTQ-FTICR mass spectrometer. No remaining protein was observed during the run. FIG. 15C shows the base peak chromatogram from bovine Ribonuclease B obtained after deglycosylation. The trapped column was analyzed using a 1200 Agilent HPLC at 1.2 mL/min connected on-line to an LTQ-FTICR mass spectrometer.

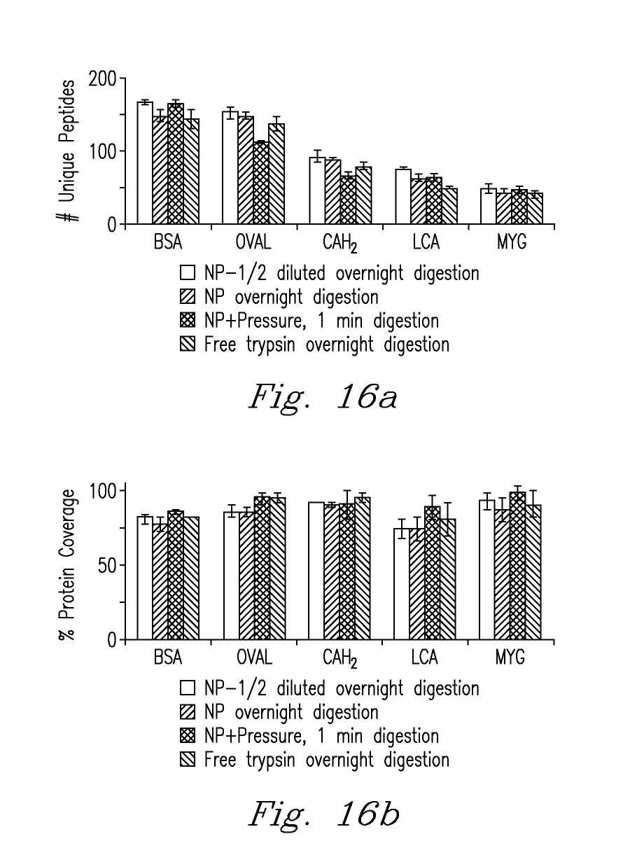

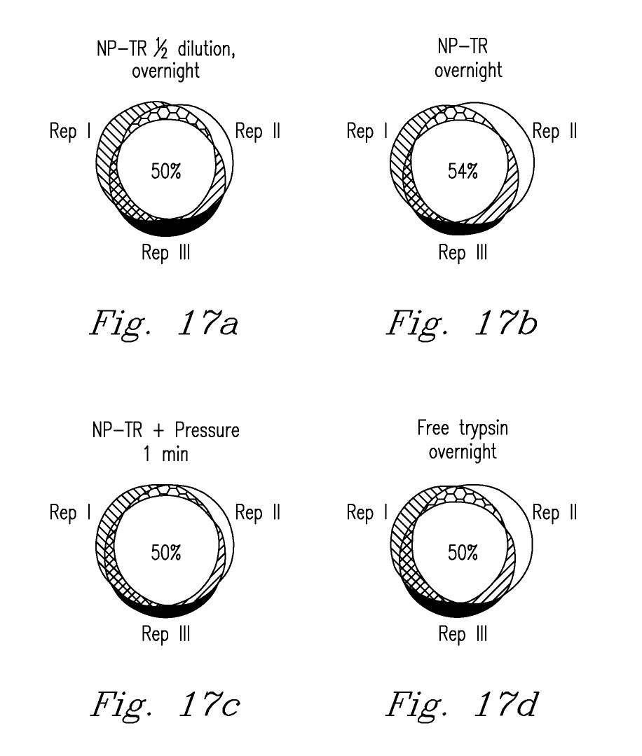

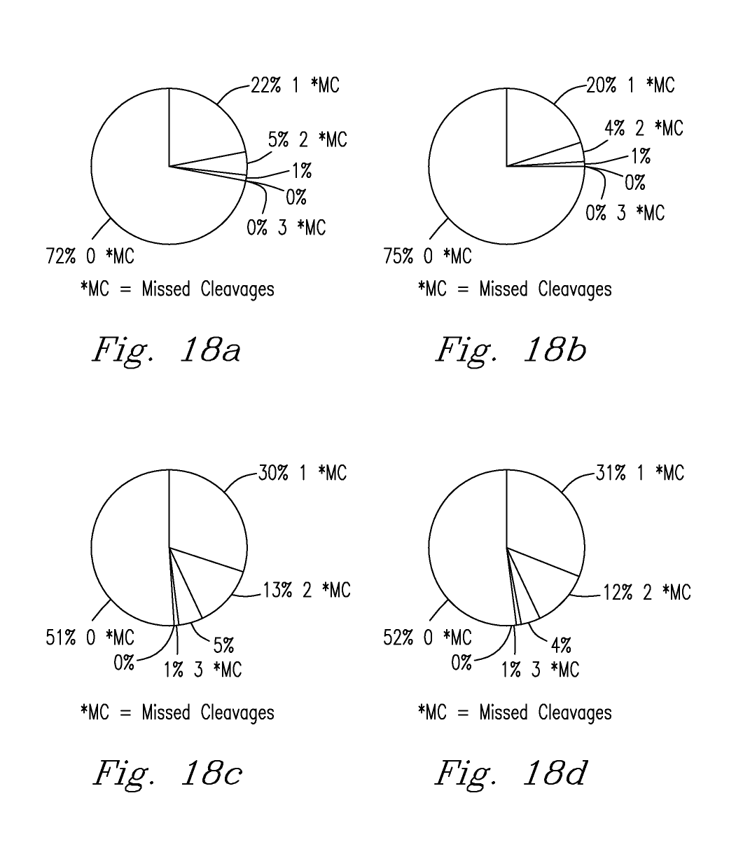

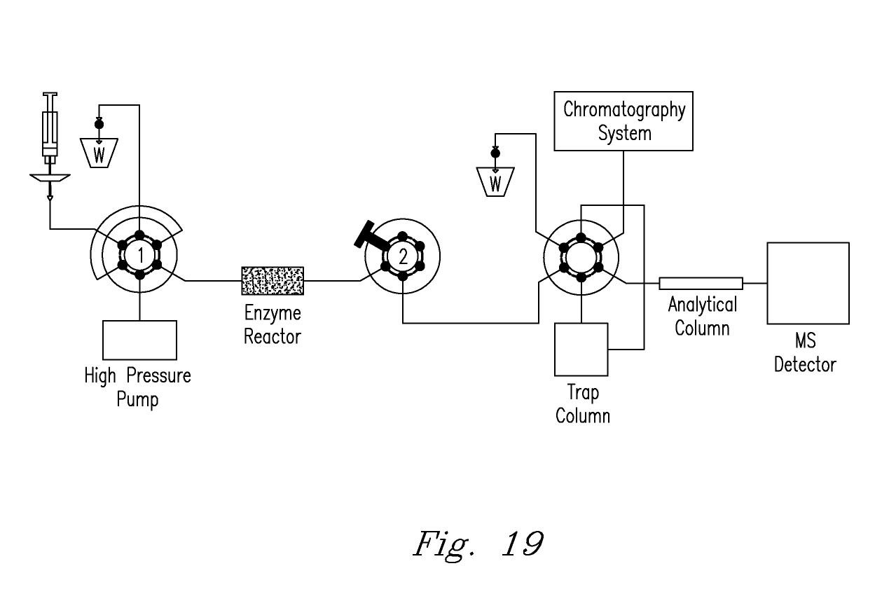

In addition to the aforementioned embodiments, in recent, years, proteomics has enormously expanded its applicability to many different scientific fields, such as biomedicine, bioterrorism, bioenergy, forensics, and food science and as the number of applications increase, the demand for higher throughput also increases. Several efforts, have aimed to increase the throughput in LC-MS analyses. One such promising effort involves the use of immobilized proteases. Immobilized proteases possess the inherent advantage of increased enzyme-substrate ratios and enhancement of the enzyme stability. Recently, highly active and stable enzyme-immobilized nanobiocomposites have been developed based on various nanobiocatalytic approaches. Various nanostructured materials, such as mesoporous materials, carbon nanotubes, nanofibers, and nanoparticles (NPs), have been used as solid supports for enzyme immobilization. One key advantage in using nanostructured materials is that their fundamental properties offer higher surface area to volume ratios. We have explored the use of magnetic NPs as enzyme immobilization agents for proteomic applications by applying an enzyme-coating protocol that can provide a highly stable, active, and reusable nanobiocatalytic system. To expedite the protein digestion, we have used enzyme-coated magnetic NPs in combination with pressure cycling technology (PCT). This novel technology platform that combines PCT and enzyme-coated magnetic NPs for rapid and efficient protein digestions was evaluated using digestions of a single protein or complex protein mixtures, and its digestion performance was compared with traditional in-solution digestions using free trypsin. We characterized the digestions in terms of reproducibility, protein coverage in terms of the number of unique peptides identified, stability across a long period of time, and tentative reusability. FIG. 16a shows a comparison of digestion performances for EC-TR/NPs and free trypsin overnight, and EC-TR/NPs pressure-assisted digestion. A five-standard protein mixture consisting of BSA, ovalbumin, myoglobin, carbonic anhydrase, and lactoglobulin was used for the experiment. FIGS. 16 (a) and 16(b) show histograms comparing the number of unique peptides and percentage of protein coverage for three technical replicates. FIGS. 17 a-d show Venn diagrams showing the peptide overlap between three technical digestion replicates. FIGS. 18 a-d show pie charts showing the percentage of missed cleavages for the different digestion methods. Looking at the reproducibility on the peptide identifications across technical replicates in all cases, ˜50% of the peptides were identified in the three replicates except in the case for of EC-TR/NPs combined with PCT where the number of peptides observed across all the technical replicates ramps up to over 60% (FIG. 16 ab). High reproducibility in the case of the PCT digestion in 1 min can be explained by the extreme accuracy of the pressure controller on the Barocylcer™, which enables an extremely efficient mixing and denaturation of the sample proteins under the pressure cycles.

FIG. 19 shows a configuration for an embodiment that allows for high pressure enzymatic reactions using immobilized enzymes, for example, proteins are first loaded into the system trap on the column pack with immobilized trypsin or pepsin. Then Valve 2 is closed and the system is pressurized for 30 seconds. When the digestion is finished, peptides are loaded into a C-18 trap column and the LC-MS analysis is perform. This system would also allow for multiplex enzymatic reactions if several columns packed with different enzymes each are setup in serial or parallel.

This system allows for a variety of alterations and modifications which are of value of any particular practitioner. By utilizing a variety of valves and sections a single sample can in a reasonable period of time be taken, treated, tested, and passed on for further testing in a variety of ways. These methods include but are not limited to HPLC, ESI, MS/MS analysis. In addition, the present invention allows for various treatment steps to take place in a rapid pressurized step at various locations thus allowing for stepwise analysis of a single sample. This provides a variety of advantages over the prior art which as has been discussed previously is subject to a variety of limitations based principally upon the slow rates of digestion of other applications.

While various preferred embodiments of the invention are shown and described, it is to be distinctly understood that this invention is not limited thereto but may be variously embodied to practice within the scope of the following claims. From the foregoing description, it will be apparent that various changes may be made without departing from the spirit and scope of the invention as defined by the following claims.

What is claimed is:

1. A method for selectively performing macromolecular fragmentation in an on-line system characterized by co-applying pressure and at least one preselected agent to a preselected material to obtain a processed sample in a period of time less than 30 minutes and performing a subsequent processing act to said processed sample without removal of said processed sample from said system.

2. The method of claim 1 wherein said preselected agents include chemicals, enzymes, microwaves, sound, ultrasound, heat, light, and combinations thereof.

3. The method of claim 1 wherein said agent is an enzyme.

4. The method of claim 3 wherein said an enzyme, this can be immobilized in a solid support or free in solution.

5. The method of claim 1 wherein said preselected period of time is between 5 seconds and 180 seconds.

6. The method of claim 1 wherein said pressure is provided in a pressure cycle ranging between 0.5 psi to 100 kpsi.

7. The method of claim 1 wherein said preselected materials are selected from the group consisting of proteins, protein macromolecules, peptides of a preselected length, organic molecules, and inorganic molecules.

8. The method of claim 7 wherein said preselected materials are present in a solid support.

9. The method of claim 7 wherein said preselected materials are present in a gel matrix.

10. The method of claim 1 further comprising the step of: treating said preselected material with isotopes in addition to said pressure and preselected agent, to create a preselected mark on said processing sample.

11. An on-line system for proteomic analysis characterized by at least two valves in a sample preparation device that fragments a protein sample using pressure in combination with a preselected agent selected from the group consisting of chemicals, enzymes, microwaves, sound, ultrasound, heat, light and combinations thereof.

12. The system of claim 11 further compromising a trapping device in between the two valves capable to trap biomolecules or other preselected agents

13. The system of claim 12 further comprising an analytical instrument.

14. The system of claim 13 wherein said sample preparation device is operative coupled to said analytical instrument.

15. The system of claim 14 further comprising an isotope in addition to said pressure and said preselected agent.

16. The system of claim 15 wherein said analytical instrument is a high pressure liquid chromatography (LC) system with a pressurized sample loop.

17. The system of claim 16 wherein said analytical instrument further comprises a mass spectrometry instrument.

18. A method for performing on-line proteomics comprising the steps of: combining a sample and an enzyme; and subjecting them to a pressure, for preselected a period of time to create a treated sample.

19. The method of claim 18 wherein said preselected time is a period of time less than 30 minutes.

20. The method of claim 18 wherein said pressure varies between 0 to 35 kpsi.

21. The method of claim 18 further comprising the step of analyzing said treated sample in the on-line analytical device.

22. The method of claim 21 wherein said on-line analytical device is a high pressure liquid chromatography (LC) system with a pressurized sample loop.

23. The method of claim 18 wherein said analytical device is a mass spectrometry instrument.
